(19) United States
(12) Patent Application Publication
(10) Pub. No.: US 2021/0369202 A1
(43) Pub. Date: Dec. 2, 2021

(54) IMPLANTABLE MEDICAL DEVICE WITH TEMPERATURE SENSOR

(71) Applicant: BIOTRONIK SE & Co. KG, Berlin (DE)

(72) Inventors: Dirk Muessig, West Linn, OR (US); Ravi Kiran Kondama Reddy, Portland, OR (US); R. Hollis Whittington, Portland, OR (US); Thomas Doerr, Berlin (DE)

(73) Assignee: BIOTRONIK SE & Co. KG, Berlin (DE)

(21) Appl. No.: 17/328,125

(22) Filed: May 24, 2021

Related U.S. Application Data
(60) Provisional application No. 63/029,720, filed on May 26, 2020.

(30) Foreign Application Priority Data
Sep. 15, 2020 (EP) 20196093.7

(51) Int. Cl. A61B 5/00; A61B 5/0205; G16H 20/40; G16H 20/30; G16H 40/67

(52) U.S. Cl. CPC A61B 5/686; A61B 5/4836; A61B 5/02055; A61B 5/318; G16H 20/30; G16H 40/67; G16H 20/40

(57) ABSTRACT

Implantable medical device for stimulating a human or animal heart comprises a processor, a memory unit, a stimulation unit configured to stimulate a cardiac region of a heart, a detection unit configured to detect an electrical signal of the same heart, and a temperature sensor for sensing a body temperature. In operation, the device performs the following steps: a) causing the temperature sensor to repeatedly sense a body temperature of a person to whom the implantable medical device is implanted: b) storing body temperature values obtained in step a) in the memory unit; c) performing a statistical analysis on at least a subset of the stored body temperature values to calculate at least one of a body temperature reference value, a variation of the sensed body temperature values and a variability of the sensed body temperature values; and d) performing at least one defined task with the calculated values.

### CROSS-REFERENCE TO RELATED APPLICATIONS

This patent application claims the benefit of and priority to co-pending European Patent Application No. EP 20196093.7, fled Sep. 15, 2020, and U.S. Provisional Patent Application No. 63/029,720, filed May 26, 2020, which are hereby incorporated by reference in their entireties.

### TECHNICAL FIELD

The present invention relates to an implantable medical device for stimulating a human or animal heart according to the preamble of claim 1, to an arrangement comprising such an implantable medical device according to the preamble of claim 7, to a method for calibrating a temperature sensor of an implantable medical device according to the preamble of claim 11, and to a computer program product according to the preamble of claim 13.

### BACKGROUND

An increase in body temperature is an early indicator for infection, e.g., for a viral infection such as influenza or COVID-19. In case of COVID-19, the available clinical data shows that patients with heart diseases appear to have a higher mortality rate compared to patients without such diseases.

Active cardiac implants are capable of collecting an implant temperature which directly corresponds to the body temperature of a person to whom the implant has been implanted. However, the on-board temperature sensors of such active cardiac implants are not calibrated to the patient body temperature.

It would be generally possible to calibrate a temperature sensor of an implantable medical device prior to its implantation. However, such external calibration is not feasible for medical devices that are already implanted in patients. For such implants, an external temperature sensor would be necessary to determine the patient's body temperature. This information could then be used to calibrate the temperature sensor of the implantable medical device. However, an external temperature sensor would be necessary for such procedure.

The present disclosure is directed toward overcoming one or more of the above-mentioned problems, though not necessarily limited to embodiments that do.

### SUMMARY

It is an object of the present invention to provide an implantable medical device with a temperature sensor, wherein the temperature sensor can be calibrated without the need of additional external devices.

At least this object is achieved with an implantable medical device having the claim elements of claim 1. Such an implantable medical device serves for stimulating a human or animal heart. It comprises a processor, a memory unit, a stimulation unit, a detection unit, and a temperature sensor. The stimulation unit serves for stimulating a cardiac region of a human or animal heart. The detection unit serves for detecting an electrical signal of the same heart. The temperature sensor serves for sensing a body temperature or a temperature of the implantable medical device which directly corresponds to the body temperature of the patient to whom the implantable medical device has been implanted.

According to the presently claimed invention, the memory unit comprises a computer-readable program that causes the processor to perform the steps explained in the following steps when executed on the processor:
a) causing the temperature sensor to repeatedly sense a body temperature of a person to whom the implantable medical device is implanted;
b) storing body temperature values obtained in step a) in the memory unit;
c) performing a statistical analysis on at least a subset of the stored body temperature values to calculate at least one of a body temperature reference value, a variation of the sensed body temperature values and a variability of the sensed body temperature values; and
d) performing the task of adding an absolute value of the variety or the variability of the sensed body temperature values to the calculated body temperature reference value or subtracting an absolute value of the variety or the variability of the sensed body temperature values from the calculated body temperature reference value to obtain an adjusted body temperature reference value and outputting the adjusted body temperature reference value or comparing an actual body temperature value with the adjusted body temperature reference value.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The idea behind the present invention shall subsequently be explained in more detail by referring to the embodiments shown in the figures. Herein:

FIG. 1 shows an implantable medical device in accordance with the present invention implanted in a patient and in communication with an external evaluation unit;

FIG. 2 shows a schematic view of an implantable medical device in accordance with the present invention; and

FIG. 3 shows a schematic view of an evaluation unit in accordance with the present invention.

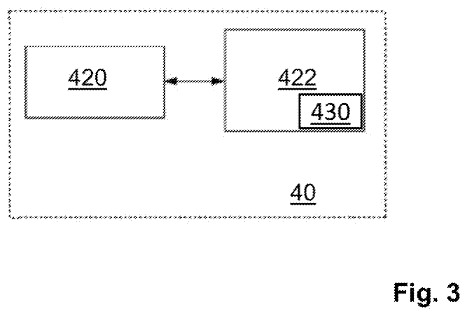

### DETAILED DESCRIPTION

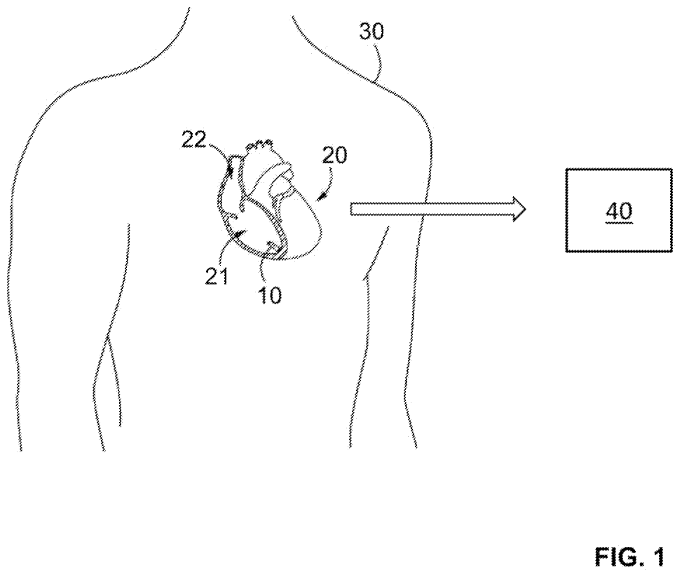

FIG. 1 illustrates the implantable medical device 10 implanted in a human patient 30 (although it could also be implanted in an anima) and attached to the heart 20. The heart 20 includes a right ventricle 21 and a right atrium 22 (and corresponding left ventricle and atrium—now shown). The implantable medical device 10 communicates with an evaluation unit 40 positioned outside the patient's body.

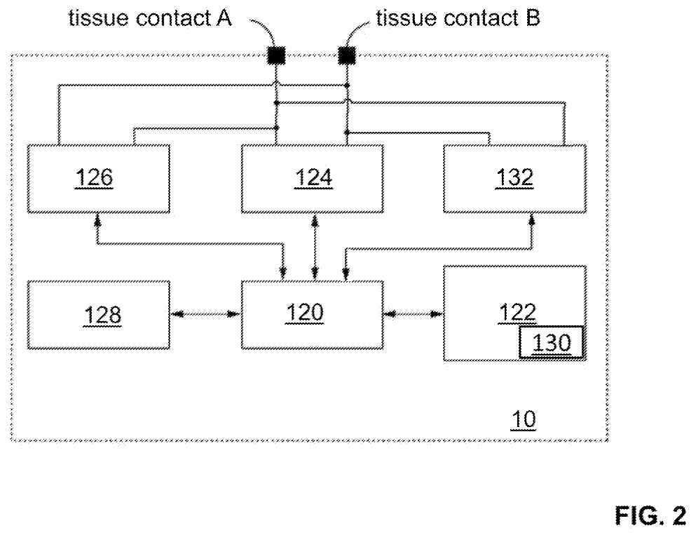

FIG. 2 illustrates the implantable medical device 10 that serves for stimulating a human or animal heart 20. The implantable medical device 10 is connected to the heart 20 at tissue contacts A and B, and comprises a processor 120, a memory unit 122, a stimulation unit 124, a detection unit 126, and a temperature sensor 132. The stimulation unit 124 serves for stimulating a cardiac region of a human or animal heart 20. The detection unit 126 serves for detecting an electrical signal of the same heart 20. The temperature sensor 132 serves for sensing a body temperature or a temperature of the implantable medical device 10 which directly corresponds to the body temperature of the patient 30 to whom the implantable medical device 10 has been implanted.

According to the presently claimed invention, the memory unit 122 comprises a computer-readable program 130 that causes the processor 120 to perform the steps explained in the following when executed on the processor 120.

First, the temperature sensor 132 is caused to sense the body temperature of the person to whom the implantable medical device 10 is implanted. In this context, the temperature sensor 132 is caused to sense the body temperature a plurality of times so that a plurality of time-dependent body temperature values is obtained. The obtained body temperature values are stored in the memory unit 122.

Afterwards, a statistical analysis on at least a subset of the stored body temperature values, in particular on all of the stored body temperature values, is performed. This statistical analysis serves for calculating a body temperature reference value and/or a variation of the sensed body temperature values and or a variability of the sensed body temperature values.

The calculated statistical parameters are then used to be output, to adjust the previously measured, calculated or determined temperature value or to be compared with an actually measured body temperature value. To be more specific, the computer-readable program 130 causes the processor 120 to perform at least one of the tasks explained in the following.

One of the possible tasks is to output the body temperature reference value and/or the variation of the sensed body temperature values and/or the variability of the sensed body temperature values. Then, these values can be used for further analyses or for documentation purposes.

Another possible task is to compare an actual body temperature value with the previously calculated body temperature reference value. In doing so, it is particularly simple to detect an increase in body temperature and thus a potential infection of the patient carrying the medical device in an implanted state. Thus, the calculated body temperature reference value serves as an internal calibration of the temperature sensor 132 of the implantable medical device 10. Even though the determination of the body temperature reference value does not enable an absolute temperature calibration of the temperature sensor 132, it guarantees a relative calibration so that future discrepancies from the calculated body temperature reference value can be easily and exactly detected.

Another possible task is to compare a predetermined expectancy value of temperature variations with the variation of the sensed body temperature values. By such a comparison, a general body temperature trend can be observed and evaluated with respect to the predetermined expectancy value (e.g., an expected temperature variation of a standard population). In doing so, patient-specific temperature variations, either normal or suspicious with respect to an infection, can be detected.

Another possible task is to compare a predetermined expectancy value of temperature variability with a variability of the sensed body temperature values. Such a comparison serves for the detection of intra-day or inter-day variances of the patient's body temperature and can help identifying future temperature deviations from the body temperature reference value as significant (i.e., being indicative for a possible infection) or as insignificant (lying within an expected range of variability). Furthermore, by evaluating the variability of the sensed body temperature, it is possible to evaluate temperature sensor 132 inaccuracies and to consider such inaccuracies for future body temperature evaluations.

Another possible task is to add an absolute value of the variety or of the variability of the sensed body temperature values to the calculated body temperature reference value or, alternatively, to subtract an absolute value of the variety or of the variability of the sensed body temperature values from the calculated body temperature reference value. In doing so, an adjusted body temperature reference value is obtained. This adjusted body temperature reference value can then be output or can be used to compare an actual body temperature value with the adjusted body temperature reference value. By using such an adjusted body temperature reference value, the sensitivity of the detection of a suspiciously increased body temperature can be increased or decreased, as desired. If an absolute value of the variety or of the variability is added to the body temperature reference value, it is less likely that a slight increase in body temperature will result in exceeding a threshold being indicative for a suspiciously increased body temperature, if the threshold is based on the adjusted body temperature reference value. Likewise, the probability that an actually sensed body temperature exceeds a threshold based on an adjusted body reference temperature value is higher if the adjusted body temperature reference value is calculated by subtracting an absolute value of the variety or of the variability of the sensed body temperature values from the previously calculated body temperature reference value.

In an embodiment, the statistical analysis comprises calculating a mean value or a median value of at least a subset of the stored body temperature values, in particular of all of the stored body temperature values. This mean value or this median value is then used as body temperature reference value.

In an embodiment, the statistical analysis comprises calculating an exponential moving average value of at least a subset of stored temperature values, in particular of all of the stored body temperature values. An exponential moving average is a type of moving average that places a greater weight and significance on the most recent data points or more specifically the most recent temperature values. This exponential moving average value is then used as body temperature reference value.

In an embodiment, the statistical analysis comprises calculating an exponential moving median value of at least a subset of stored temperature values, in particular of all of the stored body temperature values. An exponential moving median is a type of moving median that places a greater weight and significance on the most recent data points or more specifically the most recent temperature values. This exponential moving median value is then used as body temperature reference value.

In an embodiment, the statistical analysis comprises calculating a standard deviation of at least a subset of the stored body temperature values, in particular of all of the stored body temperature values. Such a standard deviation can be used as the variety of the sensed body temperature values.

In an embodiment, the statistical analysis comprises fitting at least a subset of the stored body temperature values, in particular all stored body temperature values, by a function and calculating a function parameter, hi an embodiment, the function is a function describing a line. In such a case, the function parameter is, e.g., the slope of the line. Such a slope is typically indicative for a temperature variation with time. In an embodiment, the function is a first-order exponential function and the function parameter is a coefficient or an exponent of the function.

The subset of the stored body temperature values is in an embodiment, chosen such that it comprises different body temperature values of a single day. In an embodiment, the subset of the stored body temperature values used for the statistical analysis is chosen such that it comprises body temperature values of different days. In an embodiment, the subset of the body temperature values used for the statistical analysis is chosen such that it comprises body temperature values of at least 2 days, in particular at least 3 days, in particular at least 4 days, in particular at least 5 days, in particular at least 6 days, in particular at least 7 days.

In an embodiment, the subset of the stored body temperature values used for the statistical analysis is chosen such that it comprises body temperature values of 1 to 14 days, in particular of 2 to 13 days, in particular of 3 to 12 days, in particular of 4 to II days, in particular of 5 to 10 days, in particular of 6 to 9 days, in particular of 7 to 8 days. In an embodiment, the body temperature values used for the statistical analysis originate from consecutive days.

In an embodiment, the computer-readable program 130 causes the processor 120 to sense the body temperature a plurality of times per day. E.g., the body temperature can be sensed 2 to 12 times a day. in particular 3 to 11 times a day, in particular 4 to 10 times a day, in particular 5 to 9 times a day, in particular 6 to 8 times a day. In an embodiment, the body temperature is sensed every hour, i.e., 24 times a day. In an embodiment, the temperature is sensed every 30 minutes, i.e., 48 times a day. In an embodiment, the body temperature is sensed in an interval lying between every 15 minutes and every 2 hours, in particular between every 30 minutes and every 1.5 hours, in particular between every 45 minutes and every hour.

In an embodiment, the computer-readable program 130 causes the processor 120 to sense the body temperature for a plurality of days, in particular 2 to 14 days, in particular 3 to 13 days, in particular 4 to 12 days, in particular 5 to 11 days, in particular 6 to 10 days, in particular 7 to 9 days.

In an embodiment, the computer-readable program 130 causes the processor 120 to perform the statistical analysis on body temperature values sensed on at least 3 different days, in particular at least 4 days, in particular at least 5 days, in particular at least 6 days, in particular at least 7 days. In an embodiment, the body temperature values used for the statistical analysis are sensed on a number of different days lying in a range of 3 to 14 days, in particular of 4 to 13 days, in particular of 5 to 12 days, in particular of 6 to 11 days, in particular of 7 to 10 days, in particular of 8 to 9 days.

In an embodiment, the computer-readable program 130 causes the processor 120 to output an alert if the actual body temperature value exceeds the body temperature reference value or the adjusted body temperature reference value by a predetermined value. In an embodiment, this predetermined value lies in a range from 0.3 to 2.0° C., in particular from 0.4 to 1.9° C., in particular from 0.5 to 1.8° C., in particular from 0.6 to 1.7° C., in particular from 0.7 to 1.6° C. in particular from 0.8 to 1.5° C., in particular from 0.9 to 1.4° C., in particular from 1.0 to 1.3° C., in particular from 1.1 to 1.2° C.

Such an alert can be used as indication that a risk of infection of the patient carrying the implantable medical device 10 in an implanted state is given. Medical staff, in particular a medical doctor, evaluating the patient's data can then inform the patient that he or she might be infected and can take appropriate countermeasures. Alternatively, the alert on a potential infection (e.g., a potential influenza infection or a potential COVID-19 infection) can be automatically processed and communicated to the patient.

In an aspect, the present invention relates to an arrangement comprising an implantable medical device 10 for stimulating human or animal heart 20 and an evaluation unit 40 separate from the implantable medical device 10. The implantable medical device 10 comprises a first processor 120, a first memory unit 122, a stimulation unit 124, a detection unit 126, a temperature sensor 132, and a data communication unit 128. The stimulation unit 124 serves for stimulating a cardiac region of a human or animal heart 20. The detection unit 126 serves for detecting an electrical signal of the same heart 20. The temperature sensor 132 serves for sensing a body temperature.

The first memory unit 122 of the arrangement comprises a first computer-readable program 130 that causes the first processor 120 to perform the steps explained the following when executed on the first processor.

First, the processor 120 causes the temperature sensor 132 to repeatedly sense a body temperature of the person to whom the implantable medical device 10 has been implanted. Thus, a plurality of body temperature values is obtained.

These body temperature values are then transmitted via the data communication unit 128 to the evaluation unit 40 located outside body of the person to whom the implantable medical device 10 is implanted.

The evaluation unit 40 comprises a second processor 420 and a second memory unit 422. The second memory unit 422 comprises a second computer-readable program 430 that causes the second processor 420 to perform the steps explained in the following when executed on the second processor 420.

First, the transmitted body temperature values are stored in the second memory unit 422.

Then, a statistical analysis is performed on at least a subset of the stored body temperature values, in particular on all stored body temperature values. This statistical analysis serves for calculating a body temperature reference value and/or a variation of the sensed body temperature values and/or a variability of the sensed body temperature values.

Afterwards, the evaluation unit 40 is caused to perform at least one of the tasks explained in the following.

One of the possible tasks is to output the body temperature reference value and/or the variation of the sensed body temperature values and/or the variability of the sensed body temperature values.

Another possible task is to compare an actual body temperature value with the previously calculated body temperature reference value.

Another possible task is to compare a predetermined expectancy value of temperature variations with the variation of the sensed body temperature values.

Another possible task is to compare a predetermined expectancy value of temperature variability with a variability of the sensed body temperature values.

Another possible task is to add an absolute value of the variety or of the variability of the sensed body temperature values to the calculated body temperature reference value or, alternatively, to subtract an absolute value of the variety or of the variability of the sensed body temperature values from the calculated body temperature reference value. In doing so, an adjusted body temperature reference value is obtained. This adjusted body temperature reference value can then be output or can be used to compare an actual body temperature value with the adjusted body temperature reference value.

Thus, the arrangement serves for the same purpose as the implantable medical device 10 described above. However, the data evaluation and further use of the evaluated data is not directly performed within the implantable medical device 10, but rather outside in an external evaluation unit 40. This approach requires less energy consumption of the implantable medical device 10 than evaluating the data within the implantable medical device 10. Since the energy resources of an implantable medical device 10 are very limited (in particular in case of miniaturized implantable medical devices), it is generally favorable to perform energy consuming calculation tasks outside the implantable medical device 10. In such a case, additional energy is necessary for the data transmission, the data transmission requires, however, typically much less energy than calculations and data manipulation within the implantable medical device 10.

All embodiments explained above with respect to the evaluation unit 40 of the implantable medical device 10 can be transferred to the first evaluation unit 40 and/or the second evaluation unit 40 of the described arrangement.

In an embodiment, the data communication unit 128 serves for transferring data to the evaluation unit 40 in a wireless manner. All standard data transmission protocols are appropriate for such a wireless data communication.

In an embodiment, the first computer-readable program 130 causes the first processor 120 to transmit the body temperature values at least once a day, in particular exactly once a day, in particular 2 to 12 limes a day, in particular 3 to 11 times a day. in particular 4 to 10 times a day, in particular 5 to 9 times a day, in particular 6 to 8 times a day. In an embodiment, the data is transmitted every hour, i.e., 24 times a day. In an embodiment, the data is transmitted every 30 minutes, i.e., 48 times a day. In an embodiment, the data is transmitted in an interval lying between every 15 minutes and every 2 hours, in particular between every 30 minutes and every 1.5 hours, in particular between every 45 minutes and every hour. In an embodiment, each body temperature value is transmitted directly after sensing it.

In an embodiment, the evaluation unit 40 is located remote from the implantable medical device 10. To give an example, the evaluation unit 40 can be located within a medical center and can be arranged to receive temperature and other medical data from a plurality of implantable medical devices. In such a case, the data communication unit 128 of the implantable medical device 10 typically transfers the body temperature values to an external local data communication unit 128 which then serves for further data transmission to the evaluation unit 40.

In an aspect, the present invention relates to a method for calibrating a temperature sensor 132 of an implantable medical device 10. Such an implantable medical device 10 serves for stimulating a human or animal heart 20. It comprises a processor 120, a memory unit 122, a stimulation unit 124, a detection unit 126. and a temperature sensor 132. The stimulation unit 124 serves for stimulating a cardiac region of a human or animal heart 20. The detection unit 126 serves for detecting an electrical signal of the same heart 20. The temperature sensor 132 serves for sensing a body temperature or a temperature of the implantable medical device 10 which directly corresponds to the body temperature of the patient to whom the implantable medical device 10 has been implanted.

The method comprises the steps explained in the following.

First, body temperature values are stored in the memory unit 122. The body temperature values are provided by the temperature sensor 132. They reflect the body temperature of a person carrying the implantable medical device 10 in an implant state at different time points.

Afterwards, a statistical analysis on at least a subset of the stored body temperature values, in particular on all of the stored body temperature values, is performed. This statistical analysis serves for calculating a body temperature reference value.

This body temperature reference value is then used as expected regular body temperature value for calibrating the temperature sensor 132.

In an embodiment, the method further comprises performing a statistical analysis on at least a subset of the stored body temperature values, in particular on all of the stored body temperature values, to calculate at least one of a variation of the stored body temperature values and a variability of the stored body temperature values. In this context, an absolute value of the variation or the variability of the sensed body temperature values is added to or subtracted from the calculated body temperature reference value to obtain an adjusted calculated body temperature reference value. The adjusted calculated body temperature reference value replaces then the calculated body temperature reference value during further data analysis.

In an aspect, the present invention relates to a computer program product comprising computer-readable code that causes a processor 120 to perform the steps explained in the following w hen executed on the processor 120.

In a first step, body temperature values obtained from a temperature sensor 132 of an implantable medical device 10 are stored in a memory unit 122 of the implantable medical device 10 or of an evaluation unit 40 located outside the body of a person to whom the implantable medical device 10 has been implanted.

Then, a statistical analysis is performed on at least a subset of the stored body temperature values, in particular on all stored body temperature values. This statistical analysis serves for calculating a body temperature reference value and/or a variation of the sensed body temperature values and/or a variability of the sensed body temperature values.

Afterwards, the evaluation unit 40 is caused to perform at least one of the tasks explained in the following.

One of the possible tasks is to output the body temperature reference value and/or the variation of the sensed body temperature values and or the variability of the sensed body temperature values.

Another possible task is to compare an actual body temperature value with the previously calculated body temperature reference value.

Another possible task is to compare a predetermined expectancy value of temperature variations with the variation of the sensed body temperature values.

Another possible task is to compare a predetermined expectancy value of temperature variability with a variability of die sensed body temperature values.

Another possible task is to add an absolute value of the variety or of the variability of the sensed body temperature values to the calculated body temperature reference value or, alternatively, to subtract an absolute value of the variety or of the variability of the sensed body temperature values from the calculated body temperature reference value. In doing so, an adjusted body temperature reference value is obtained. This adjusted body temperature reference value can then be output or can be used to compare an actual body temperature value with the adjusted body temperature reference value.

In an aspect, the present invention relates to a method for sensing a body temperature of a person in need of such sensing, wherein the method comprises the steps explained in the following.

First, the body temperature of a person to whom an implantable medical device 10 has been implanted is repeatedly sensed with a temperature sensor 132 of the implantable medical device 10. In this context, the implantable medical device 10 serves for stimulating a human or animal heart 20. It comprises, besides the temperature sensor 132, a processor 120, a memory unit 122, a stimulation unit 124, and a detection unit 126. The stimulation unit 124 serves for stimulating a cardiac region of a human or animal heart 20. The detection unit 126 serves for detecting an electrical signal of the same heart 20. The temperature sensor 132 serves for sensing a body temperature or a temperature of the implantable medical device 10 which directly corresponds to the body temperature of the patient to whom the implantable medical device 10 has been implanted.

The body temperature is sensed a plurality of times so that a plurality of time-dependent body temperature values is obtained. The obtained body temperature values are stored in the memory unit 122.

Afterwards, a statistical analysis on at least a subset of the stored body temperature values, in particular on all of the stored body temperature values, is performed. This statistical analysis serves for calculating a body temperature reference value and/or a variation of the sensed body temperature values and/or a variability of the sensed body temperature values.

The calculated statistical parameters are then used to be output, to adjust the previously measured, calculated or determined temperature value or to be compared with an actually measured body temperature value To be more specific, at least one of the tasks explained the following is performed.

One of the possible tasks is to output the body temperature reference value and/or the variation of the sensed body temperature values and or the variability of the sensed body temperature values.

Another possible task is to compare an actual body temperature value with the previously calculated body temperature reference value.

Another possible task is to compare a predetermined expectancy value of temperature variations with the variation of the sensed body temperature values.

Another possible task is to compare a predetermined expectancy value of temperature variability with a variability of the sensed body temperature values.

Another possible task is to add an absolute value of the variety or of the variability of the sensed body temperature values to the calculated body temperature reference value or, alternatively, to subtract an absolute value of the variety or of the variability of the sensed body temperature values from the calculated body temperature reference value. In doing so, an adjusted body temperature reference value is obtained. This adjusted body temperature reference value can then be output or can be used to compare an actual body temperature value with the adjusted body temperature reference value.

In an embodiment, the method further comprises a step of indicating a risk of an infection of the person if an actual body temperature value measured with a temperature sensor 132 exceeds the body temperature reference value or the adjusted body temperature reference value by a predetermined value. In an embodiment, this predetermined value lies in a range from 0.3 to 2.0° C. in particular from 0.4 to 1.9° C., in particular front 0.5 to 1.8° C., in particular from 0.6 to 1.7° C., in particular from 0.7 to 1.6° C., in particular from 0.8 to 1.5° C., in particular from 0.9 to 1.4° C., in particular from 1.0 to 1.3° C., in particular front 1.1 to 1.2° C.

In an embodiment, the risk of infection is a risk of an influenza infection or the risk of a COVID-19 infection.

All embodiments of the implantable medical device 10 can be combined in any desired way and can be transferred either individually or in any arbitrary combination to the described arrangement, the described methods, and to the described computer program product. Likewise, all embodiments of the described arrangement can be combined in any desired way and can be transferred either individually or in any arbitrary culmination to the described implantable medical device 10, to the described methods, and to the described computer-readable program. Furthermore, all embodiments of the described methods can be combined in any desired way and can be transferred either individually or in any arbitrary combination to the respective other method, to the implantable medical device 10, 10 the arrangement, and to the computer program product. Finally, all embodiments of the described computer program product can be combined in any desired way and can be transferred either individually or in any arbitrary combination to the described implantable medical device 10, to the described arrangement, and to the described methods.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMBERS

10—implantable medical device
20—heart
21—right ventricle
22—right atrium
30—human
40—evaluation unit
120—first processor
122—first memory unit
124—stimulation unit
126—detection unit
130—first computer-readable program
132—temperature sensor
420—second processor
422—second memory unit
430—second computer-readable program

What is claimed is:

1. An implantable medical device or stimulating a human or animal heart, the device comprising a processor, a memory unit, a stimulation unit configured to stimulate a cardiac region of a human or animal heart, a detection unit configured to detect an electrical signal of the same heart, and a temperature sensor for sensing a body temperature,
wherein the memory unit comprises a computer-readable program that causes the processor to perform the following steps when executed on the processor:
a) causing the temperature sensor to repeatedly sense a body temperature of a person to whom the implantable medical device is implanted;
b) storing body temperature values obtained in step a) in the memory unit;
c) performing a statistical analysis on at least a subset of the stored body temperature values to calculate at least one of a body temperature reference value, a variation of the sensed body temperature values and a variability of the sensed body temperature values; and
d) performing the task of adding an absolute value of the variety or the variability of the sensed body temperature values to the calculated body temperature reference value or subtracting an absolute value of the variety or the variability of the sensed body temperature values from the calculated body temperature reference value to obtain an adjusted body temperature reference value and outputting the adjusted body temperature reference value or comparing an actual body temperature value with the adjusted body temperature reference value.

2. The implantable medical device according to claim 1, wherein the statistical analysis is chosen from the group consisting of calculating a mean value of at least a subset of the stored body temperature values, calculating a median value of at least a subset of the stored body temperature values, calculating an exponential moving average value of at least a subset of stored temperature values, calculating an exponential moving median value of at least a subset of stored temperature values, calculating a standard deviation of at least a subset of the stored body temperature values, fitting at least a subset of the stored body temperature values by a function and calculating a function parameter.

3. The implantable medical device according to claim 1, wherein the computer-readable program causes the processor to sense the body temperature multiple times per day.

4. The implantable medical device according to claim 1, wherein the computer-readable program causes the processor to sense the body temperature for multiple days.

5. The implantable medical device according to claim 1, wherein the computer-readable program causes the processor to perform the statistical analysis on the body temperature values sensed on at least three different days.

6. The implantable medical device according to claim 1, wherein the computer-readable program causes the processor to output an alert if the actual body temperature value exceeds the body temperature reference value or the adjusted body temperature reference value by a predetermined value.

7. An arrangement, comprising an implantable medical device for stimulating a human or animal heart and an evaluation unit separate from the implantable medical device wherein the implantable medical device comprises a first processor, a first memory unit, a stimulation unit configured to stimulate a cardiac region of a human or animal heart, a detection unit configured to detect an electrical signal of the same heart, a temperature sensor for sensing a body temperature, and a data communication unit, wherein the first memory unit comprises a first computer-readable program that causes the first processor to perform the following steps when executed on the first processor:
a) causing the temperature sensor to repeatedly sense a body temperature of a person to whom the implantable medical device is implanted;
b) transmitting body temperature values obtained in step a) via the data communication unit to the evaluation unit located outside a body of the person to whom the implantable medical device is implanted;
and in that the evaluation unit comprises a second processor and a second memory unit, wherein the second memory unit comprises a second computer-readable program that causes the second processor to perform the following steps when executed on the second processor:
c) storing the transmitted body temperature values in the second memory unit;
d) performing a statistical analysis on at least a subset of the stored body temperature values to calculate at least one of a body temperature reference value, a variation of the sensed body temperature values and a variability of the sensed body temperature values; and
e) performing the task of adding an absolute value of the variety or the variability of the sensed body temperature values to the calculated body temperature reference value or subtracting an absolute value of the variety or the variability of the sensed body temperature values from the calculated body temperature reference value to obtain an adjusted body temperature reference value and outputting the adjusted body temperature reference value or comparing an actual body temperature value with the adjusted body temperature reference value.

8. The arrangement according to claim 7, wherein the data communication unit is arranged and designed to transfer data to the evaluation unit in a wireless manner.

9. The arrangement according to claim 7, wherein the first computer-readable program causes the first processor to transmit the body temperature values at least once a day.

10. The arrangement according to claim 7, wherein the evaluation unit is located remote from the implantable medical device.

11. A method for calibrating a temperature sensor of an implantable medical device for stimulating a human or animal heart, the implantable medical dev ice comprising a processor, a memory unit, a stimulation unit configured to stimulate a cardiac region of a human or animal heart, a detection unit configured to detect an electrical signal of the same heart, and a temperature sensor for sensing a body temperature.
the method comprising the following steps:
a) storing body temperature values provided by the temperature sensor in the memory unit, the body temperature values reflecting a body temperature at different time points;
b) performing a statistical analysis on at least a subset of the stored body temperature values to calculate a body temperature reference value; and
c) calibrating the temperature sensor by using the calculated body temperature reference value as expected regular body temperature value,
wherein the method further comprises performing a statistical analysis on at least a subset of the stored body temperature values to calculate at least one of a variation of the stored body temperature values and a variability of the stored body temperature values, wherein an absolute value of the variation or the variability of the sensed body temperature values is added to or subtracted from the calculated body temperature reference value to obtain an adjusted calculated body temperature reference value replacing the calculated body temperature reference value.

12. A computer program product comprising computer-readable code that causes a processor to perform the following steps when executed on the processor;
a) storing body temperature values obtained from a temperature sensor of an implantable medical device in a memory unit of the implantable medical device or of an evaluation unit located outside a body of the person to whom the implantable medical device is implanted;
b) performing a statistical analysis on at least a subset of the stored body temperature values to calculate at least one of a body temperature reference value, a variation of the sensed body temperature values and a variability of the sensed body temperature values; and
c) performing the task of adding an absolute value of the variety or the variability of the sensed body temperature values to the calculated body temperature reference value or subtracting an absolute value of the variety or the variability of the sensed body temperature values from the calculated body temperature reference value to obtain an adjusted body temperature reference value and outputting the adjusted body temperature reference value or comparing an actual body temperature value with the adjusted body temperature reference value.

13. A method for sensing a body temperature of a person in need of such sensing, the method comprising the following steps:
a) repeatedly sensing a body temperature of a person to whom an implantable medical device is implanted with a temperature sensor of the implantable medical device, wherein the implantable medical device serves for stimulating a human or animal heart and further comprises a processor, a memory unit, a stimulation unit configured to stimulate a cardiac region of a human or animal heart, and a detection unit configured to detect an electrical signal of the same heart;
b) storing body temperature values obtained in step a) in the memory unit;
c) performing a statistical analysis on at least a subset of the stored body temperature values to calculate at least one of a body temperature reference value, a variation of the sensed body temperature values and a variability of the sensed body temperature values; and
d) performing the task of adding an absolute value of the variety or the variability of the sensed body temperature values to the calculated body temperature reference value or subtracting an absolute value of the variety or the variability of the sensed body temperature values from the calculated body temperature reference value to obtain an adjusted body temperature reference value and outputting the adjusted body temperature reference value or comparing an actual body temperature value with the adjusted body temperature reference value.

14. The method according to claim 13, further comprising indicating a risk of an infection of the person if an actual body temperature value measured with the temperature sensor exceeds the body temperature reference value or the adjusted body temperature reference value by a predetermined value.

15. The method according to claim 14, wherein the predetermined value lies in a range from 0.3 to 2.0° C.
